(19) United States
(12) Patent Application Publication
(10) Pub. No.: US 2023/0182303 A1
(43) Pub. Date: Jun. 15, 2023

(54) SURGICAL ROBOTIC SYSTEM INSTRUMENT ENGAGEMENT AND FAILURE DETECTION

(71) Applicant: Covidien LP, Mansfield, MA (US)

(72) Inventor: Brian K. Wells, Killingworth, CT (US)

(21) Appl. No.: 17/923,963

(22) PCT Filed: May 27, 2021
(86) PCT No.: PCT/US21/34467

§ 371 Date: Nov. 8, 2022

Related U.S. Application Data
(60) Provisional application No. 63/035,882, filed on Jun. 8, 2020.

(51) Int. Cl. B25J 9/16; A61B 34/30

(52) U.S. Cl. CPC B25J 9/1674; A61B 34/30; A61B 2090/066

(57) ABSTRACT

A surgical robotic arm includes an instrument having a coupler rotatable about a longitudinal axis, the coupler including: a drive screw; a drive nut threadably coupled to the drive screw, the drive nut movable along the longitudinal axis in response to rotation of the drive screw; and a drive member coupled to the drive nut and movable in response to movement of the drive nut. The surgical robotic arm also includes an instrument drive unit having: a motor configured to engage the coupler and rotate about the longitudinal axis to rotate the coupler and the drive screw; one or more sensors configured to measure one or more properties of the motor; and a controller coupled to the sensor(s) and the motor. The controller is configured to control the motor based on the property of the motor.

### BACKGROUND

### 1. Technical Field

The present disclosure generally relates to a surgical robotic system having one or more modular arm carts each of which supports a robotic arm, and a surgical console for controlling the carts and their respective arms. More particularly, the present disclosure is directed to a system and method for detecting engagement between an instrument and an instrument drive unit of the robotic arm as well as detecting mechanical failure in the instrument using sensors within the instrument drive unit.

### 2. Background of Related Art

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Since instruments are couplable to the robotic arm there is a need to monitor whether the instrument is properly engaged to the robotic arm and that the instrument is functioning properly to prevent damaging the instrument and/or injuring the patient.

### SUMMARY

According to one embodiment of the present disclosure, a surgical robotic arm is disclosed. The surgical robotic arm includes: an instrument having a coupler rotatable about a longitudinal axis, the coupler including a drive screw; a drive nut threadably coupled to the drive screw, the drive nut movable along the longitudinal axis in response to rotation of the drive screw; and a drive member coupled to the drive nut and movable in response to movement of the drive nut. The surgical robotic arm also includes: an instrument drive unit having a motor configured to engage the coupler and rotate about the longitudinal axis to rotate the coupler and the drive screw; one or more sensors configured to measure one or more properties of the motor; and a controller coupled to the sensor(s) and the motor. The controller is configured to control the motor based on the property of the motor.

According to one aspect of the above embodiment, the controller may be further configured to detect mechanical failure of the instrument based on the at least on property of the motor. The controller may be further configured to perform a comparison of the property of the motor to a threshold. The controller may be further configured to stop the motor based on the comparison of the property of the motor to the threshold. The sensor(s) may be a current sensor configured to measure a current draw of the motor, a torque sensor configured to measure torque output by the motor, or an angle sensor configured to measure an angle of rotation of the motor.

According to another aspect of the above embodiment, the sensors may include a current sensor configured to measure a current draw of the motor, a torque sensor configured to measure torque output by the motor, and an angle sensor configured to measure an angle of rotation of the motor. The controller may be further configured to stop the motor in response to any of the current draw, the torque, or the angle of rotation of the motor exceeding a corresponding threshold. The controller may be further configured to detect engagement of the motor with the coupler. The controller may be configured to detect the engagement of the motor with the coupler by: operating the motor until a torque threshold is achieved by the motor; and comparing torque measured by the sensor to a target torque value. The motor may be operated at a constant speed and in a dithering pattern.

According to another embodiment of the present disclosure, a method for controlling a surgical robotic arm is disclosed. The method includes: coupling an instrument to an instrument drive unit having a motor configured to engage a coupler of the instrument; and measuring at least one property of the motor through the sensor. The method also includes: performing, at a controller, a comparison of the at least one property of the motor to a threshold; and detecting, at the controller, mechanical failure of the instrument based on the comparison.

According to one aspect of the above embodiment, the method may further include: stopping the motor based on the comparison of the at least one property of the motor to the threshold. The method may further include: measuring a current draw of the motor; measuring torque output by the motor; and measuring an angle rotation of the motor. The method may also include stopping the motor in response to any of the current draw, the torque, or the angle of rotation of the motor exceeding a corresponding threshold.

According to another aspect of the above embodiment, the method may further include detecting engagement of the motor with the coupler. The detection of the engagement may further include operating the motor until a torque threshold is achieved by the motor; and comparing torque measured by the sensor to a target torque value. Operating the motor may include operating the motor at a constant speed in a dithering pattern.

According to a further embodiment of the present disclosure, a surgical robotic arm is disclosed. The surgical robotic arm includes an instrument having: a coupler rotatable about a longitudinal axis, the coupler including a drive screw; a drive nut threadably coupled to the drive screw, the drive nut movable along the longitudinal axis in response to rotation of the drive screw; and a drive member coupled to the drive nut and movable in response to movement of the drive nut. The surgical robotic arm also includes an instrument drive unit having: a motor configured to engage the coupler and rotate about the longitudinal axis to rotate the coupler and the drive screw; one or more sensors configured to measure at least one property of the motor; and a controller coupled to the sensor(s) and the motor. The controller is configured to control the motor based on the at least one property of the motor and to detect engagement of the motor with the coupler.

According to one aspect of the above embodiment, the controller may be further configured to detect mechanical failure of the instrument based on the at least on property of the motor. The controller may be configured to detect the engagement of the motor with the coupler by: operating the motor at a constant speed and in a dithering pattern until a torque threshold is achieved by the motor; and comparing torque measured by the at least one sensor to a target torque value.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a perspective view of an instrument drive unit and a surgical instrument according to an embodiment of the present disclosure;

FIG. 6 is a perspective view, with parts separated, of the instrument drive unit and the surgical instrument shown in FIG. 5 according to an embodiment of the present disclosure;

FIG. 7 is a rear perspective view of the surgical instrument for use with the robotic surgical assembly of FIGS. 5 and 6;

FIG. 8 is a perspective view of drive assemblies of the surgical instrument of FIG. 7;

FIG. 9 is a cross-sectional view of the surgical instrument, as taken through 9-9 of FIG. 7;

FIG. 10 is a top, perspective view of an end effector, according to an embodiment of the present disclosure, for use in the surgical robotic system of FIG. 1;

FIG. 11 is a method for determining a mechanical fault in the surgical instrument according to an embodiment of the present disclosure; and

FIG. 12 is a method for determining engagement between the instrument drive unit and the surgical instrument according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

As will be described in detail below, the present disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

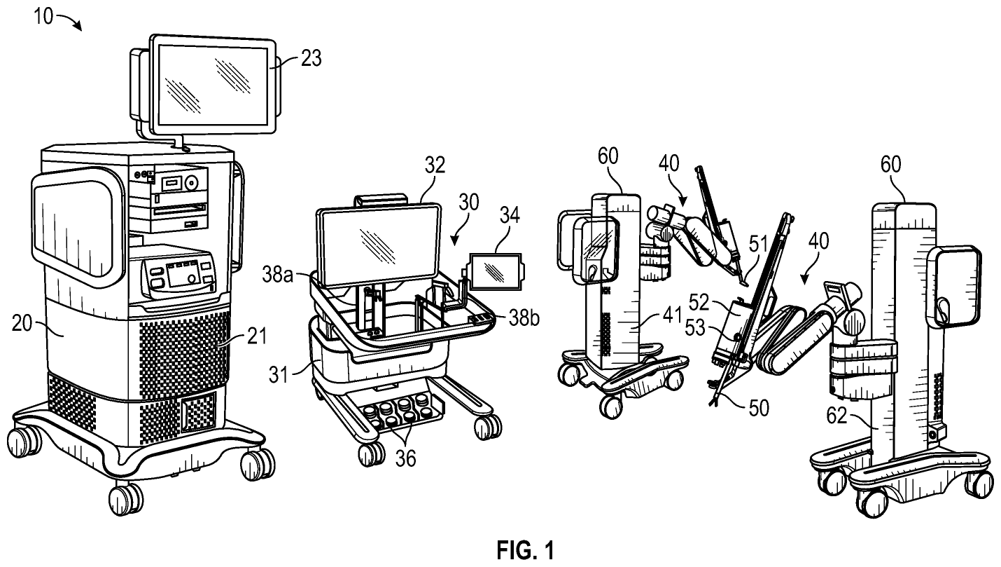

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endscope camera 51, configured to provide a video feed for the user. In further embodiments, the surgical instrument 50 may be an electrosurgical forceps configured to seal tissue by compression tissue between jaw members and applying electrosurgical current thereto. In yet further embodiments, the surgical instrument 50 may be a surgical stapler including a pair of jaws configured to grasp and clamp tissue whilst deploying a plurality of tissue fasteners, e.g., staples, and cutting stapled tissue.

One of the robotic arms 40 may include a camera 51 configured to capture video of the surgical site. The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of hand controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the hand controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

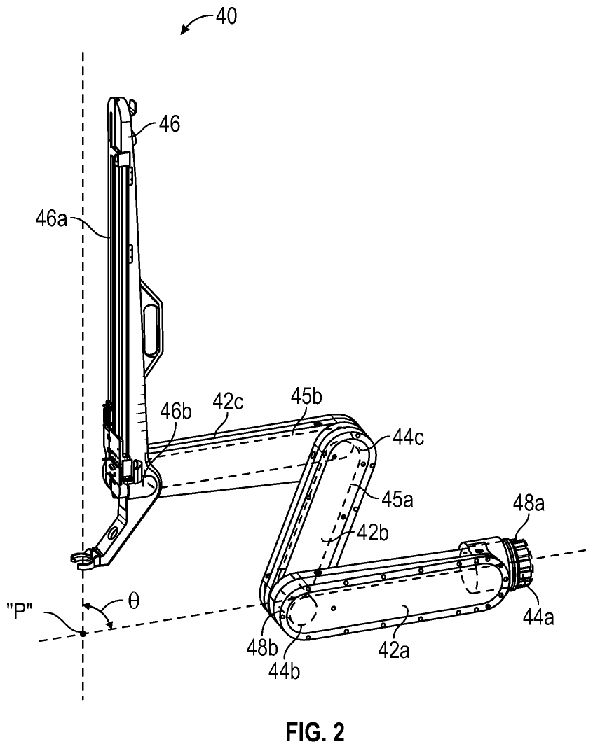

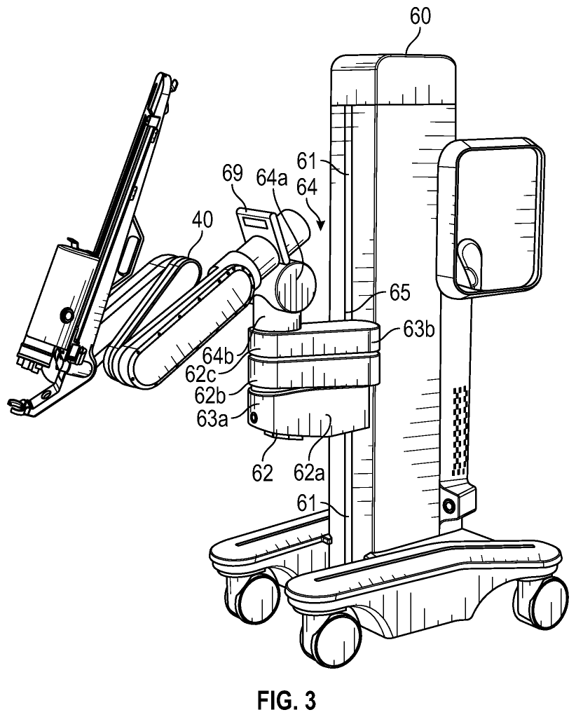

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference to FIG. 2, the robotic arm 40 also includes a holder 46 defining a second longitudinal axis and configured to receive an IDU 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 also includes a plurality of manual override buttons 53 disposed on the IDU 52 and the setup arm 62, which may be used in a manual mode. The user may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

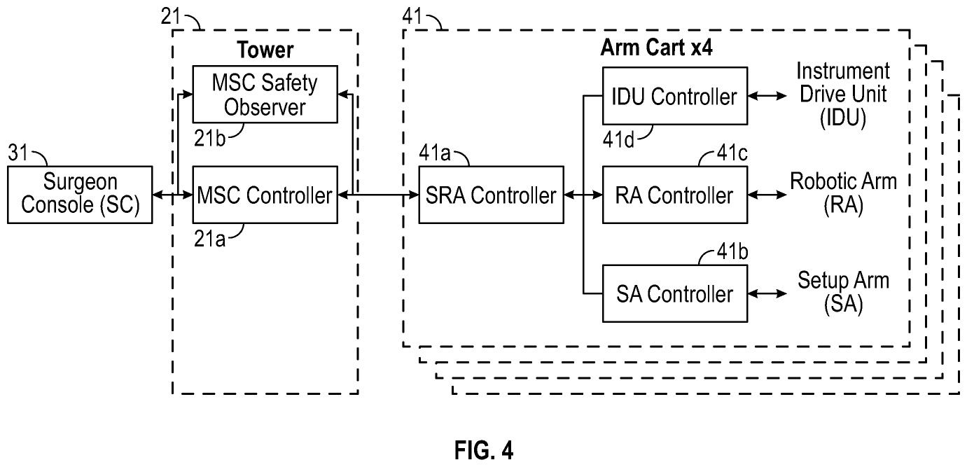

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the hand controller controlling the robotic arm 40, e.g., the hand controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the hand controller 38a is then scaled by a scaling function executed by the controller 21a. In embodiments, the coordinate position is scaled down and the orientation is scaled up by the scaling function. In addition, the controller 21a also executes a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

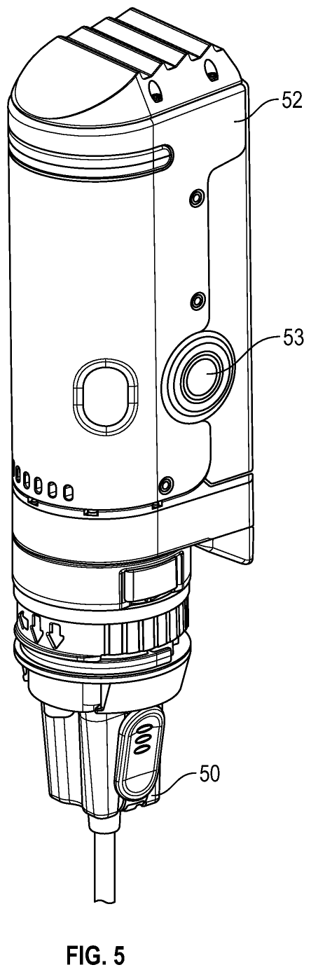

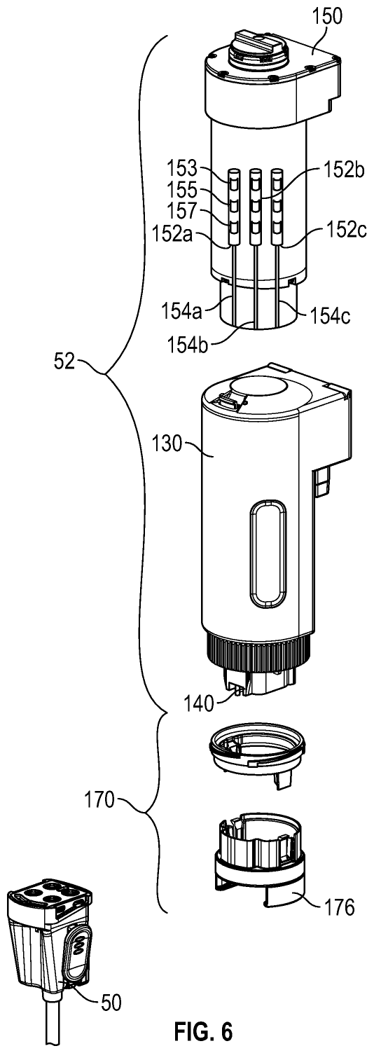

With reference to FIGS. 5 and 6, the IDU 52 is shown in more detail and is configured to transfer power and actuation forces from its motors 152a, 152b, 152c to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

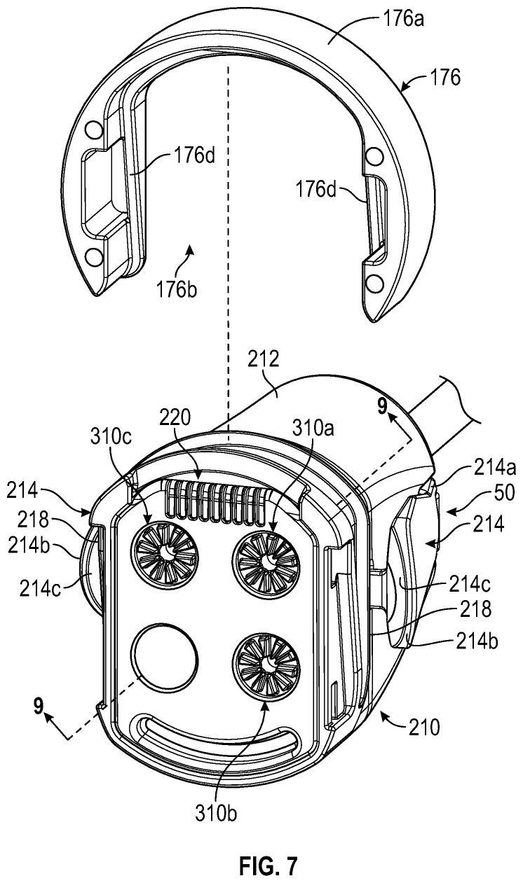

The IDU 52 includes a motor pack 150 and a sterile barrier housing 130. Motor pack 150 includes motors 152a, 152b, 152c for controlling various operations of the instrument 50. The instrument 50 is removably coupleable to IDU 52. As the motors 152a, 152b, 152c of the motor pack 150 are actuated, rotation of the drive transfer shafts 154a, 154b, 154c of the motors 152a, 152b, 152c, respectively, is transferred to the respective proximal couplers 310a, 310b, 310c of the drive assemblies 300a, 300b, 300c (FIG. 7) of the instrument 50.

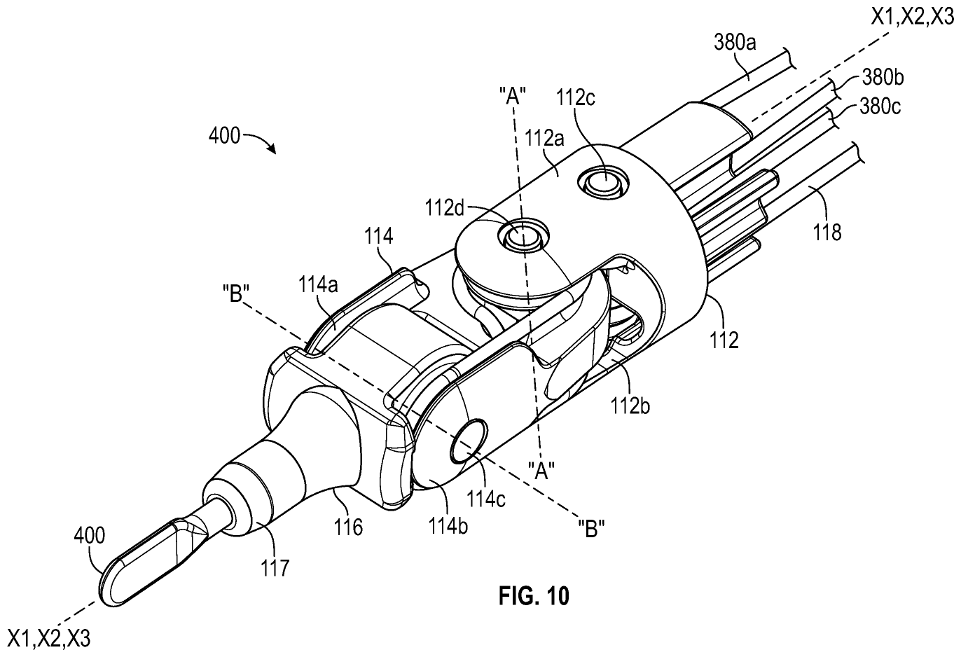

The instrument 50 may have an end effector 400 (FIG. 10) secured to a distal end thereof. The instrument 50 is configured to transfer rotational forces/movement supplied by the IDU 52 (e.g., via the motors 152a, 152b, 152c of the motor pack 150) into longitudinal movement or translation of the drive members 380a, 380b, 380c to effect various functions of the end effector 400.

With reference to FIGS. 7-9, the instrument 50 includes a housing assembly 210 with a housing 212 defining at least one cavity or bore 212a, 212b, 212c, 212d therein which is configured to receive a respective drive assembly 300a, 300b, 300c and a power cable 118 therein. In accordance with the present disclosure, each bore 212a, 212b, 212c of the housing 212 is configured to operatively support a respective drive assembly 300a, 300b, and 300c therein and the bore 212d is configured to operatively support a power cable 118 therein.

As illustrated in FIGS. 7-9, each bore 212a, 212b, 212c of the housing 212 defines a respective longitudinally extending groove or channel 213a, 213b, 213c therein. Each channel 213a, 213b, 213c is configured to slidingly accept a rail or tab 353a, 353b, 353c extending radially from a respective drive nut 350a, 350b, 350c of a respective drive assembly 300a, 300b, 300c.

When the instrument 50 is connected to the IDU 52, the proximal couplers 310a, 310b, 310c of the drive assemblies 300a, 300b, 300c of the instrument 50 come into registration with and are connected to respective drive transfer shafts 154a, 154b, 154c within the IDU 52 (FIGS. 5 and 6) to couple the respective drive assemblies 300a, 300b, 300b to respective motors 152a, 152b, 152c of the IDU 52.

The housing 212 of the housing assembly 210 of the instrument 50 also supports an electrical connector 220 (FIG. 7) configured for selective connection to the plug 140 of the IDU 52 (FIGS. 5 and 6) of the IDU 52. The instrument 50 may include electronics, including, and not limited to, a memory (for storing identification information, usage information, and the like), wired or wireless communication circuitry for receiving and transmitting data or information. The IDU 52 may be configured to permit passage or routing of a dedicated electrocautery cable (e.g., cable 118) or the like for use and connection to an electrosurgical based electromechanical surgical instrument (e.g., for ablation, coagulation, sealing, etc.). The electrical connector 220 may include and is not limited to conductive connectors, magnetic connectors, resistive connectors, capacitive connectors, Hall sensors, reed switches or the like.

With continued reference to FIGS. 7-9, the housing assembly 210 of the instrument 50 houses a plurality of drive assemblies, shown as drive assemblies 300a, 300b, 300c. In the illustrated embodiment, the instrument 50 includes three drive assemblies 300a, 300b, 300c; however, the instrument 50 may include more (e.g., four, five, or six) or fewer (e.g., two) drive assemblies without departing from the scope of the present disclosure.

Each drive assembly 300a, 300b, 300c includes a respective proximal coupler 310a, 310b, 310, proximal bearing 320a, 320b, 320c, drive screw 340a, 340b, 340c, drive nut 350a, 350b, 350c, biasing element 370a, 370b, 370c, and drive member (e.g., a drive rod or drive cable) 380a, 380b, 380c. The proximal coupler 310a, 310b, 310c of each drive assembly 300a, 300b, 300c is configured to meshingly engage with respective drive transfer shafts 154a, 154b, 154c coupled to respective motors of the IDU 52. In operation, rotation of the drive transfer shafts 154a, 154b, 154c of the motors 152a, 152b, 152c results in corresponding rotation of respective proximal coupler 310a, 310b, 310c of respective drive assembly 300a, 300b, 300c.

The proximal coupler 310a, 310b, 310c of each drive assembly 300a, 300b, 300c is keyed to or otherwise non-rotatably connected to a proximal end of a respective drive screw 340a, 340b, 340c. Accordingly, rotation of the proximal coupler 310a, 310b, 310c results in a corresponding rotation of a respective drive screw 340a, 340b, 340c.

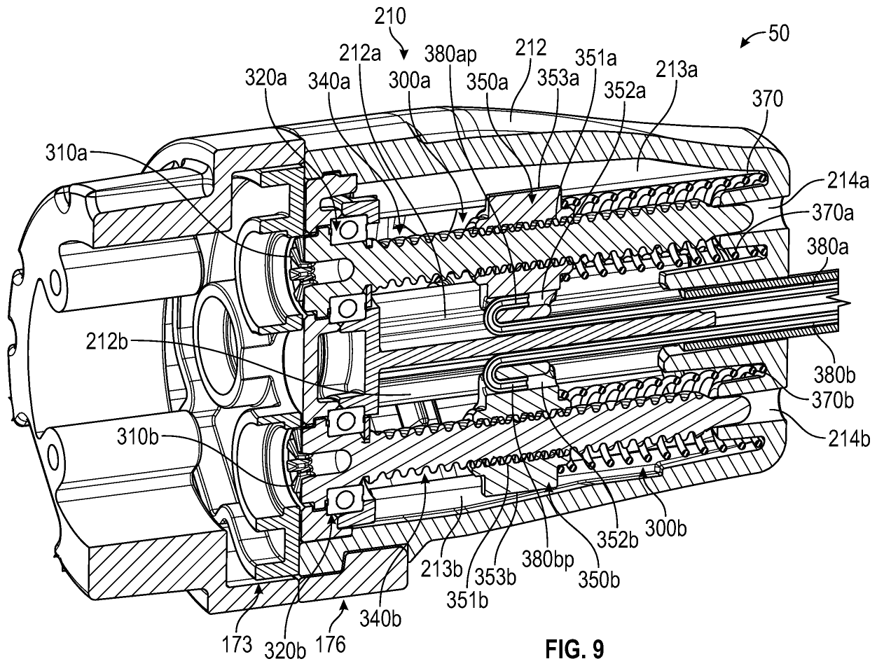

Each proximal bearing 320a, 320b, 320c is disposed about a proximal portion of a respective drive screw 340a, 340b, 340c adjacent a proximal end of the housing 212 of the housing assembly 210. A distal end or tip of each drive screw 340a, 340b, 340c may be rotatably disposed or supported in a respective recess 214a, 214b, 214c defined in a distal end of the housing 212 (see FIG. 9).

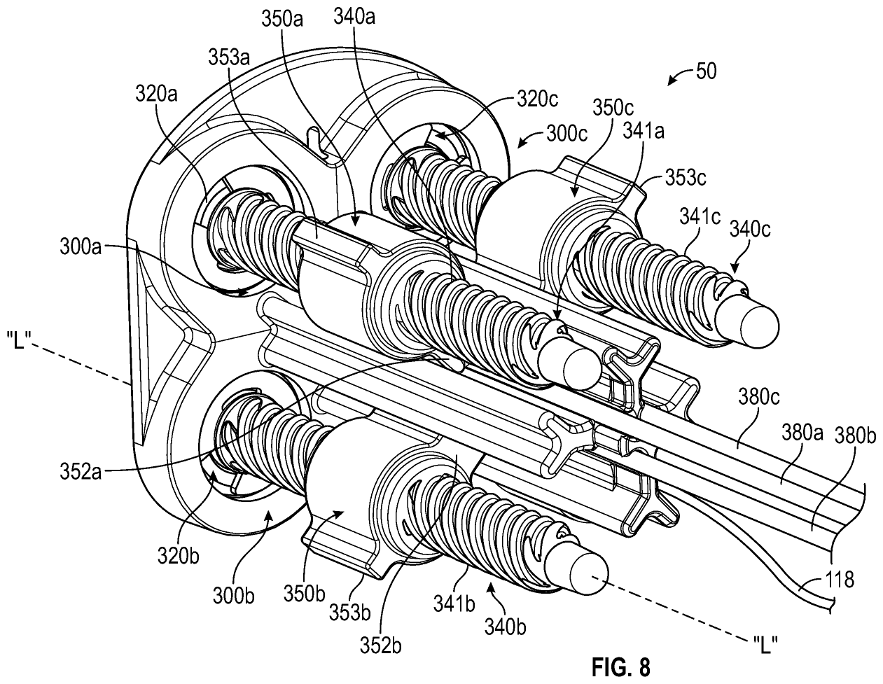

Each of the drive screws 340a, 340b, 340c includes a threaded body or shaft portion 341a, 341b, 341c, and defines a longitudinal axis “L-L” extending through a radial center thereof (see FIG. 8). In use, rotation of the proximal coupler 310a, 310b, 310c, as described above, results in rotation of a respective drive screw 340a, 340b, 340c about longitudinal axis “L-L”, in a corresponding direction and rate of rotation.

Each of the drive nuts 350a, 350b, 350c includes a threaded aperture 351a, 351b, 351c extending longitudinally therethrough, which is configured to mechanically engage the threaded shaft portion 341a, 341b, 341c of a respective drive screw 340a, 340b, 340c. Each drive nut 350a, 350b, 350c is configured to be positioned on a respective drive screw 340a, 340b, 340c in a manner such that rotation of the drive screw 340a, 340b, 340c causes longitudinal movement or translation of the respective drive nut 350a, 350b, 350c. Moreover, rotation of the proximal coupler 310a, 310b, 310c in a first direction (e.g., clockwise) causes the respective drive nut 350a, 350b, 350c to move in a first longitudinal direction (e.g., proximally) along the respective drive screw 340a, 340b, 340c, and rotation of the proximal coupler 310a, 310b, 310c in a second direction (e.g., counter-clockwise) causes the respective drive nut 350a, 350b, 350c to move in a second longitudinal direction (e.g., distally) with respect to the respective drive screw 340a, 340b, 340c.

Each drive nut 350a, 350b, 350c includes a retention pocket formed in an engagement tab 352a, 352b, 352c formed therein that is disposed adjacent the threaded aperture 351a, 351b, 351c thereof. Each retention pocket is configured to retain a proximal end 380ap,380bp,380cp of a respective drive member 380a, 380b, 380c, as discussed in further detail below.

Each drive nut 350a, 350c, 350c includes a tab 353a, 353b, 353c extending radially from and longitudinally along an outer surface thereof. The tab 353a, 353b, 353c of each drive nut 350a, 350b, 350c is configured to be slidably disposed in a respective longitudinally extending channel 213a, 213b, 213c formed in the bores 212a, 212b, 212c of the housing 212. The tab 353a, 353b, 353c of each drive nut 350a, 350b, 350c cooperates with a respective channel 213a, 213b, 213c of the bore 212a, 212b, 212c of the housing 212 to inhibit or prevent each drive nut 350a, 350b, 350c from rotating about longitudinal axis “L-L” as each drive screw 340a, 340b, 340c is rotated.

The engagement portions 352a, 352b, 352c of each of the drive nuts 350a, 350b, 350c includes is disposed adjacent a radially inward surface thereof, which is configured to mechanically engage or retain a proximal portion 380ap,380bp,380cp of a respective drive member 380a, 380b, 380c. In operation, as the drive nuts 350a, 350b, 350c are axially displaced along the drive screw 340a, 340b, 340c, the drive nuts 350a, 350b, 350c transmit concomitant axial translation to the drive member 380a, 380b, 380c.

A biasing element 370a, 370b, 370c, e.g., a compression spring, is configured to radially surround a respective distal portion of the threaded shaft portion 341a, 341b, 341c of each drive screw 340a, 340b, 340c. Each biasing element 370a, 370b, 370c is interposed between a respective drive nut 350a, 350b, 350c and a distal surface of the housing 212 of the housing assembly 210.

Each drive member 380a, 380b, 380c extends distally from a respective drive nut 350a, 350b, 350c, through a respective central bore or channel 212a, 212b, 212c of the housing 212 of the housing assembly 210, and is configured to mechanically engage a portion of a surgical instrument, e.g., a portion or component of end effector 400, of the instrument 50. Additionally, power cable 118 extends distally through central bore 212d of the housing 212 of the housing assembly 210, and is configured to electrically couple to the end effector 400.

In operation, longitudinal translation of at least one drive member 380a, 380b, 380c is configured to drive a function of the end effector 400 of the instrument 50. In embodiments, a proximal translation of drive member 380c may be configured to articulate the end effector 400 or a portion of the end effector 400 in a first direction. It is envisioned that while drive member 380c is translated in a proximal direction, drive nuts 350a and 350b are translated in a distal direction to enable corresponding translation of respective drive members 380a and 380b in a distal direction, as will be described in greater detail below. In further embodiments, a proximal translation of drive members 380a and 380b of the instrument 50 may be configured to articulate the end effector 400, or a portion of the end effector 400 in a second direction. It is envisioned that while drive members 380a and 380b are translated in a proximal direction, drive nut 350c is translated in a distal direction to enable corresponding translation of drive member 380c in a distal direction, as will be described in greater detail below.

In accordance with the present disclosure, a distal portion of at least one of the drive members 380a, 380b, 380c may include a flexible portion, while a proximal portion of the drive members 380a, 380b, 380c are rigid, such that the flexible distal portion may follow a particular path through the instrument 50. Accordingly, the biasing elements 370a, 370b, 370c may function to maintain the drive members 380a, 380b, 380c in tension to prevent slack or to reduce the amount of slack in the flexible distal portion of the drive members 380a, 380b, 380c.

During use of the instrument 50 (e.g., when motor 152a, 152b, 152c of the IDU 52, or other powered drives, are used to rotate one or more of proximal couplers 310a, 310b, 310c), rotation of a proximal coupler 310a, 310b, 310c results in a corresponding rotation of the respective drive screw 340a, 340b, 340c. Rotation of the drive screw 340a, 340b, 340c causes longitudinal translation of the respective drive nut 350a, 350b, 350c due to the engagement between the threaded portion 341a, 341b, 341c of the drive screw 340a, 340b, 340c and the threaded aperture 351a, 351b, 351c of the drive nut 350a, 350b, 350c. As discussed above, the direction of longitudinal translation of the drive nut 350a, 350b, 350c is determined by the direction of rotation of the proximal coupler 310a, 310b, 310c, and thus, the respective drive screw 340a, 340b, 340c. For example, clockwise rotation of the drive screw 340a results in a corresponding proximal translation of drive member 380a which is engaged with the drive screw 340a, clockwise rotation of the drive screw 340b results in a corresponding proximal translation of drive member 380b which is engaged with the drive screw 340b, and clockwise rotation of the drive screw 340c results in a corresponding proximal translation of drive member 380c which is engaged with the drive screw 340c. Additionally, for example, counterclockwise rotation of the drive screw 340a results in a corresponding distal translation of drive member 380a which is engaged with the drive screw 340a, counterclockwise rotation of the drive screw 340b results in a corresponding distal translation of drive member 380b which is engaged with the drive screw 340b, and counterclockwise rotation of the drive screw 340c results in a corresponding distal translation of drive member 380c which is engaged with the drive screw 340c.

Additionally, in one aspect, when one drive nut 350a, 350b, 350c, from a first drive assembly 300a, 300b, 300c, moves in a first longitudinal direction (e.g., proximally), it is envisioned that a different drive nut 350a, 350b, 350c, from a different drive assembly 300a, 300b, 300c, is forced to correspondingly move in a second, opposite longitudinal direction (e.g., distally). Such a function may be accomplished via the physical interaction between the individual drive assemblies 300a, 300b, 300c amongst each other or via control of the respective motors 152a, 152b, and 152c, as will be described in greater detail below. Such configurations function to, for example, compensate for any slack in the drive members 380a, 380b, 380c or to create a slack in drive members 380a, 380b, 380c. It is contemplated and in accordance with the present disclosure that each drive nut 350a, 350b, 350c may be independently driven.

As discussed above, each of the motors 152a, 152b, and 152c may be controlled in a corresponding manner to negate slack formation in any of drive members 380a, 380b, 380c, when another one of drive members 380a, 380b, or 380c (e.g., an opposing drive member) is translated in an opposing direction. Additionally, each of the motors 152a, 152b, and 152c may be controlled in a corresponding manner to create slack in any of drive members 380a, 380b, 380c, when another one of drive members 380a, 380b, or 380c (e.g., an opposing drive member) is translated in an opposing direction. Such corresponding control of the motors 152a, 152b, 152c ensures that the proximal translation of any of drive members 380a, 380b, or 380c is not hindered by the stationary position of an opposing drive member 380a, 380b, or 380c. For example, when motor 152c is actuated to cause proximal translation of drive nut 350c (thereby translating drive member 380c in a proximal direction), motors 152a and 152b are coordinated with motor 152c to actuate in an opposite direction to cause distal translation of respective drive nuts 350a and 350b (thereby enabling drive members 380a and 380b to be moved in a distal direction when effectively pulled in a distal direction by the opposing force of drive member 380c). Additionally, for example, when motors 152a and 152b are actuated to cause proximal translation of respective drive nuts 350a and 350b (thereby translating respective drive members 380a and 380b in a proximal direction), motor 152c is coordinated with motors 152a and 152b to actuate in an opposite direction to cause distal translation of drive nut 350c (thereby enabling drive member 380c to be moved in a distal direction when effectively pulled in a distal direction by the opposing force of drive member 380c). Additionally, for example, when motor 152a is actuated to cause proximal translation of drive nut 3 50a (thereby translating drive member 3 80a in a proximal direction), motor 152b may be coordinated with motor 152a to actuate in an opposite direction to cause distal translation of drive nut 350b (thereby enabling drive member 380b to be moved in a distal direction when effectively pulled in a distal direction by the opposing force of drive member 380a), and vice versa.

With reference to FIG. 6, each of the motors 152a, 152b, 152c includes a current sensor 153, a torque sensor 155, and an angle sensor 157. For conciseness only the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The angle sensor 157 may be any device that provides a sensor signal indicative of the number of rotations of the motor 152a, such as a mechanical encoder or an optical encoder. Parameters which are measured and/or determined by the angle sensor 157 may include speed, distance, revolutions per minute, position, and the like. The sensor signals from sensors 153, 155, 157 are transmitted to the IDU controller 41d, which then controls the motor 152a based on the sensor signals. In embodiments, additional position sensors may also be used, which include, but are not limited to, potentiometers coupled to movable components and configured to detect travel distances, Hall Effect sensors, accelerometers, and gyroscopes.

Sensor signals from the sensors 153, 155, 157 may be used to detect cable failure of the drive members 380a, 380b, 380c or any other failure of mechanical linkage components of the instrument 50. Detection of mechanical failure of any of the mechanical linkage components of the instrument 50, such as failure of drive members 380a, 380b, 380c may be used to stop continued operation of the instrument 50 to prevent damaging the instrument 50 or injuring the patient. Thus, if drive members 380a, 380b, 380c are broken, the drive nuts 350a, 350b, 350c may be continuously operated and collide with the housing 212 of the instrument 50 (FIG. 9). Thus, error detection based on the feedback of the sensors 153, 155, 157 protects the instrument 50 by limiting the travel of the drive nuts 350a, 350b, 350c.

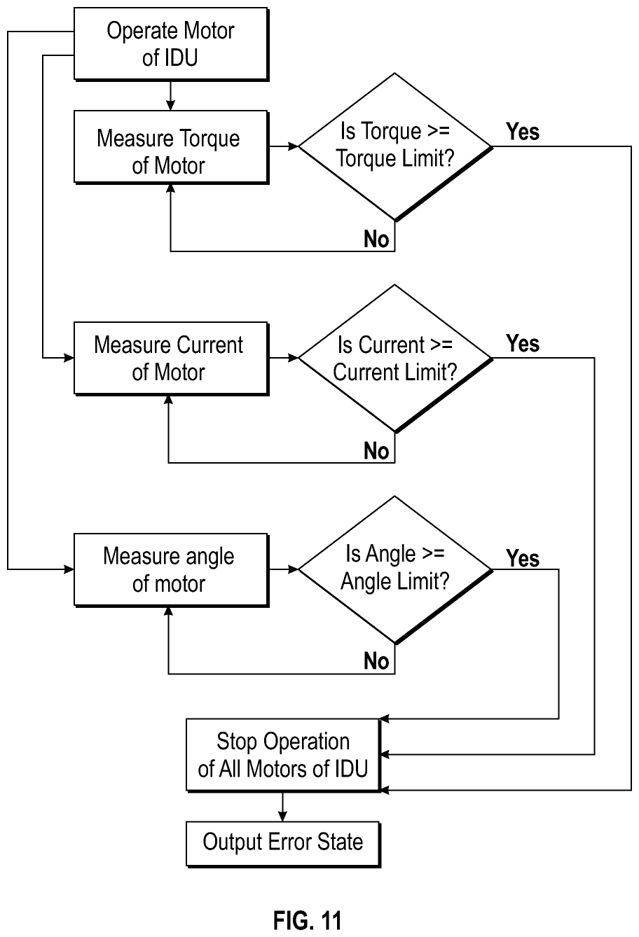

With reference to FIG. 11, the main cart controller 41a checks for errors in the IDU controller 41d and changes motor commands controlling the motor 152a in response to a fault detection by the IDU controller 41d. The IDU controller 41d is configured to detect a plurality of faults based on the feedback from the sensors 153, 155, 157. The IDU controller 41d stores in memory (not shown) a maximum torque value, maximum current value, and maximum angle value. Each of the values may be specific to the instrument 50 that is coupled to the IDU 52. Thus, the IDU controller 41d may store multiple maximum values and select a corresponding maximum value based on the connected instrument 50. During operation, the IDU controller 41d continuously compares each of the measured torque, current, and motor angle to the maximum corresponding value. If any of the measured values is equal to or exceeds the maximum value, the IDU controller 41d enters an error state. In this state, the IDU controller 41d is configured to stop operation of all of the motors 152a, 152b, 152c such that the instrument 50 is no longer operational. In embodiments, the instrument 50 may be controlled automatically such that the instrument 50 may be extracted from the patient. The IDU controller 41d is also configured to output to the main cart controller 41a the error state such that no additional commands are sent to the IDU controller 41d and the error is propagated through the system 10.

Sensor signals from the sensors 153, 155, 157 may be used to enable and detect proper coupling between the IDU 52 and the instrument 50. As noted above, each of the motors 152a, 152b, 152c, rotates corresponding drive transfer shafts 154a, 154b, 154c, which results in corresponding rotation of respective proximal coupler 310a, 310b, 310c of respective drive assembly 300a, 300b, 300c of the instrument 50. Thus, proper coupling may be accomplished and determined based on detection of a mechanical load due to engagement between each of the transfer shafts 154a, 154b, 154c and the corresponding couplers 310a, 310b, 310c. For conciseness only operation of the motor 152a, the transfer shaft 154a, and the coupler 310a are used below to describe detection of coupling between the IDU 52 and the instrument 50.

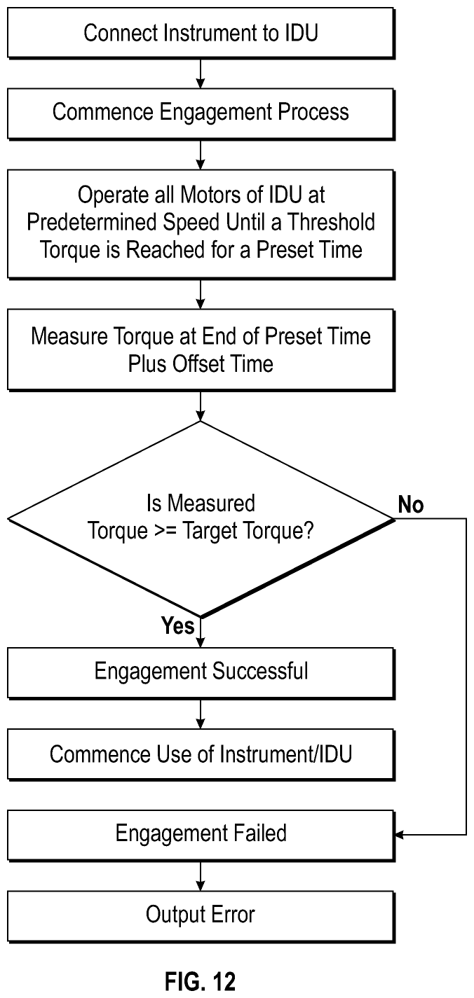

With reference to FIG. 12, initially, the instrument 50 is manually attached to the IDU 52, which then commences an engagement process controlled by the IDU controller 41d. Engagement involves activating the motor 152a to mechanically engage the coupler 310. The motor 152a is activated to ramp up torque up to a predetermined target torque value, which may be from about 0.01 Nm to about 0.1 Nm.

During engagement, the motor 152a may be rotated at a constant speed, which may be about 1 radian per second, for a predetermined period of time, which may be from about 10 ms to about 5,000 ms. In addition, during engagement the motor 152a may be activated in a dithering pattern to break the friction between the transfer shaft 154a and the coupler 310a. Dithering may include oscillating between clockwise and counterclockwise directions and/or temporarily stopping and restarting motion of the motor 152a in one or both of the directions. Dithering is performed within a predetermined torque threshold, which may be about 0.005 Nm. Dithering may be performed at a frequency of about 1 kHz.

The process also includes measuring torque imparted by the motor 152a to determine if the transfer shaft 154a engaged the coupler 310a. The motor 152a is activated during a preset ramp period to ramp up to a target torque value, which may be about 0.02 Nm. Once the target torque value is reached, the motor 152a is deactivated and the torque is released. Torque imparted by the motor 152a is measured during engagement. If after expiration of a predetermined time period the target torque is not detected, the engagement is determined to have failed and the IDU controller 41d outputs an error and stops operation of the motor 152a. The predetermined time period includes the time of the ramp trajectory and an offset value, which may be from about 1 ms to about 50 ms.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic arm comprising:
an instrument including:
a coupler rotatable about a longitudinal axis, the coupler including a drive screw;
a drive nut threadably coupled to the drive screw, the drive nut movable along the longitudinal axis in response to rotation of the drive screw; and
a drive member coupled to the drive nut and movable in response to movement of the drive nut; and
an instrument drive unit including:
a motor configured to engage the coupler and rotate about the longitudinal axis to rotate the coupler and the drive screw;
at least one sensor configured to measure at least one property of the motor; and
a controller coupled to the at least one sensor and the motor, the controller configured to control the motor based on the at least one property of the motor.

2. The surgical robotic arm according to claim 1, wherein the controller is further configured to detect mechanical failure of the instrument based on the at least on property of the motor.

3. The surgical robotic arm according to claim 2, wherein the controller is further configured to perform a comparison of the at least one property of the motor to a threshold.

4. The surgical robotic arm according to claim 3, wherein the controller is further configured to stop the motor based on the comparison of the at least one property of the motor to the threshold.

5. The surgical robotic arm according to claim 1, wherein the at least one sensor is at least one of a current sensor configured to measure a current draw of the motor, a torque sensor configured to measure torque output by the motor, or angle sensor configured to measure an angle of rotation of the motor.

6. The surgical robotic arm according to claim 1, wherein the at least one sensor includes a current sensor configured to measure a current draw of the motor, a torque sensor configured to measure torque output by the motor, an angle sensor configured to measure an angle of rotation of the motor.

7. The surgical robotic arm according to claim 6, wherein the controller is further configured to stop the motor in response to any of the current draw, the torque, or the angle of rotation of the motor exceeding a corresponding threshold.

8. The surgical robotic arm according to claim 1, wherein the controller is further configured to detect engagement of the motor with the coupler.

9. The surgical robotic arm according to claim 8, wherein the controller is configured to detect the engagement of the motor with the coupler by:
operating the motor until a torque threshold is achieved by the motor; and
comparing torque measured by the at least one sensor to a target torque value.

10. The surgical robotic arm according to claim 9, wherein the motor is operated at a constant speed and in a dithering pattern.

11. A method for controlling a surgical robotic arm, the method includes:
coupling an instrument to an instrument drive unit having a motor configured to engage a coupler of the instrument;
measuring at least one property of the motor through at least one sensor;
performing, at a controller, a comparison of the at least one property of the motor to a threshold; and
detecting, at the controller, mechanical failure of the instrument based on the comparison.

12. The method according to claim 11, further comprising:
stopping the motor based on the comparison of the at least one property of the motor to the threshold.

13. The method according to claim 12, further comprising:
measuring a current draw of the motor;
measuring torque output by the motor; and
measuring an angle rotation of the motor.

14. The method according to claim 13, further comprising:
stopping the motor in response to any of the current draw, the torque, or the angle of rotation of the motor exceeding a corresponding threshold.

15. The method according to claim 11, further comprising:
detecting engagement of the motor with the coupler.

16. The method according to claim 11, wherein detection of the engagement further includes:
operating the motor until a torque threshold is achieved by the motor; and
comparing torque measured by the at least one sensor to a target torque value.

17. The method according to claim 16, wherein operating the motor includes operating the motor at a constant speed in a dithering pattern.

18. A surgical robotic arm comprising:
an instrument including:
a coupler rotatable about a longitudinal axis, the coupler including a drive screw;
a drive nut threadably coupled to the drive screw, the drive nut movable along the longitudinal axis in response to rotation of the drive screw; and
a drive member coupled to the drive nut and movable in response to movement of the drive nut; and
an instrument drive unit including:
a motor configured to engage the coupler and rotate about the longitudinal axis to rotate the coupler and the drive screw;
at least one sensor configured to measure at least one property of the motor; and
a controller coupled to the at least one sensor and the motor, the controller configured to control the motor based on the at least one property of the motor and to detect engagement of the motor with the coupler.

19. The surgical robotic arm according to claim 18, wherein the controller is further configured to detect mechanical failure of the instrument based on the at least on property of the motor.

20. The surgical robotic arm according to claim 18, wherein the controller is configured to detect the engagement of the motor with the coupler by:
operating the motor at a constant speed and in a dithering pattern until a torque threshold is achieved by the motor; and
comparing torque measured by the at least one sensor to a target torque value.
